(19) United States
(12) Patent Application Publication
(10) Pub. No.: US 2021/0317427 A1
(43) Pub. Date: Oct. 14, 2021

(54) Optimized 3-phytase and its use in food or feed production

(71) Applicant: Clariant Produkte (Deutschland) GmbH, Frankfurt am Main (DE)

(72) Inventors: Helge JOCHENS, Baierbrunn (DE); Frank WALLRAPP, Gauting (DE); Michael HOESL, München (DE); Thomas EISELE, Eichenau (DE); Lutz ROECHER, München (DE); Gabi GEBHARDT-WEIDL, Essenbach (DE); Martin GRAEBER, Stockdorf (DE); Werner STUERMER, Inning (DE); Jörg CLAREN, Stockdorf (DE); Timothy O'CONNELL, Landsberg am Lech (DE); Martin SCHATTE, München (DE); Christoph REISINGER, Gauting (DE)

(21) Appl. No.: 17/266,335

(22) PCT Filed: Jul. 17, 2019
(86) PCT No.: PCT/EP2019/069298

§ 371 Date: Feb. 5, 2021

(30) Foreign Application Priority Data
Aug. 8, 2018 (EP) 18188107.9

(51) Int. Cl. C12N 9/16; A23K 10/14; A23K 20/189

(52) U.S. Cl. CPC C12N 9/16; A23K 20/189; A23K 10/14; C12Y 301/03008

(57) ABSTRACT

The present invention refers to an optimized 3-phytase which is a bio-efficacious phytase mutant having sequence identity of at least 95% around 3-phytase (SEQ ID NO. 1) comprising a sequence of SEQ ID NO. 2 and one or more mutations. The invention is further directed to the use of such optimized 3-phytase in the production of a food or feed product.

The present invention refers to an optimized 3-phytase which is a bio-efficacious phytase mutant having sequence identity of at least 95% around 3-phytase (SEQ ID NO. 1) comprising a sequence of SEQ ID NO.2 and one or more mutations. The invention is further directed to the use of such optimized 3-phytase in the production of a food or feed product.

### TECHNICAL BACKGROUND

Phytases are enzymes which remove phosphate from organophosphate compounds such as phytate and phytic acid, respectively. Many types of phytases are known in the art and some of them are already used as an additive to animal feed products in order to improve metabolization and the utilization of phytate naturally present within animal feed stuff such as corn, wheat and soybeans. The classification of the phytases depends on the starting position of cleavage of phosphate from the phytate molecule in the first place. 3-phytases (E.C. 3.1.3.8, also referred to as: 1-phytase; myo-inositol-hexakisphosphate 3-phosphohydrolase; phytate 1-phosphatase or phytate 3-phosphatase) start with the phosphate cleavage at position 3 of the phytate molecule. 6-phytases start with the phosphate cleavage at position 6. Depending on the type of phytase, further phosphate cleavage of other positions occurs. 3-phytases for example are able to cleave all phosphate groups from phytate.

The addition of a phytase to food or feed enables better metabolization of the natural phytate content of the food or feed, as phosphate is an important element of a human's or an animal's diet, but also leads to a decrease of remaining phosphor in the excrements. High phosphorous contents in excrements usually leads to severe limitations of its use as manure as many countries set strict limitations.

In order to overcome these drawbacks of an insufficient natural phytate metabolization and a costly artificial phosphorous enrichment of food or feed which is also known to particularly increase remaining phosphorous content in manure, phytases have already been introduced in food or feed products to increase phytate metabolization in the stomach and digestive tract.

Examples for such phytases which are currently commercially available originate from Escherichia coli, Citrobacter braakii, Buttiauxella species and Aspergillus niger.

Each of these phytase products has limitations in at least specific activity, dosage requirements, reaction time and accumulation of inhibiting intermediate compounds. First of its kind products contained fungal 3-phytases which require high enzyme dosing in order to achieve the desired effect. Currently available, commercial feed products try to overcome this effect by using 6-phytases of bacterial origin. A severe drawback of these 6-phytases is, however, an accumulation of the intermediate compound inositol-tetraphosphate which severely limits efficient phytate metabolization. In addition, phytate and its intermediates, particularly inositol-tetraphosphate, form complexes with divalent ions such as Ca²⁺, Zn²⁺ and Mg²⁺. Further, many of the phytase products known within the art require the use of a high amount of phytase to guarantee a sufficient phosphate release regarding the actual critical retention time of the food or feed in the humans or animals stomach of a maximum of 1.5 hours.

Several factors influence phytase functionality and their in vivo phytase enzyme activity in the digestive tract of humans or animals In pigs for example, feed phytase is mainly active in the stomach and upper part of the small intestine, and added phytase activity is not recovered in the ileum. In poultry for example, feed phytase activities are mainly found in the upper part of the digestive tract, including the crop, proventriculus and gizzard. For fish with a stomach for example, phytase activities are mainly in the stomach. Many factors can influence the efficiency of food or feed phytase in the gastrointestinal tract, and they can be divided into three main groups: (i) phytase related; (ii) dietary related and (iii) subject related. Phytase-related factors include type of phytase (e.g. 3- or 6-phytase; bacterial or fungal phytase origin), the pH optimum and the resistance of phytase to endogenous protease. Dietary-related factors are mainly associated with dietary phytate content, food or feed ingredient composition and food or feed processing, and total P, Ca and Na content. Subject-related factors include species, gender and age of subjects (Yueming Dersjant-Li et al., J. Sci. Food Agric., 2015 Mar. 30; 95(5): 878-896).

Food and feed, respectively, is generally produced at high temperatures, e.g., to reduce contamination, and in consequence phytases used in food and/or feed production have to be thermostable.

Therefore, there is a need for a phytase that works over a wide range of pH values and thus, is active in the stomach as well as the upper intestine and that is at the same time stable at high temperature.

The present invention fills this gap and provides 3-phytase mutants such as bacterial 3-phytase mutants comprising mutations which result in unexpected and surprising temperature and pH stability, wherein the 3-phytase is active and highly efficient in phosphate cleavage.

### SUMMARY

The present invention refers to a bio-efficacious phytase mutant such as a 3-phytase mutant having sequence identity of at least 96.25% around SEQ ID NO. 1, wherein the mutant comprises a sequence of SEQ ID NO.2 and a mutation selected from the group consisting of D149N, S189D, K190N, K191N, R210Q, L211E, L211M, K25E, K25A, G26N, G26S, N193H, N193K, N193S, N193R, D1E, P3T, G26K, K29A, T30S, T30A, T30K, D36N, Q45N, Q45R, V58E, S75C, Q76R, P107S, A112G, D120K, T129K, Q136R, Q144L, D149N, K187E, D189N, G192R, S195R, S195Y, S195T, S195N, N204I, N204T, N204S, S214A, N216D, A230T, M237L, N243K, N248K, R255H, G257S, F264Y, A275P, D276G, K295Y, K295M, Q332D, A352S, V383M, R393H, A395T and combinations thereof.

The bio-efficacious phytase mutant of the present invention comprises for example mutations D149N, S189D, K190N and K191N, or R210Q and L211M, or K25E or K25A, and G26N or G26S, and N193S or N193R; or D149N, S189D, K190N, K191N, R210Q, L211M, K25E or K25A, and G26N or G26S, and N193S or N193R.

The bio-efficacious phytase mutant of the present invention comprises for example mutations at positions 25, 26, 193, 210 and/or 211.

Further advantageous phytase mutants of the present invention showing an advantageous thermostability comprise the mutations D149N, S189D, K190N and K191N, wherein particularly advantageous phytase mutants further comprise the mutations K25E or K25A; N193H, N193S, N193R or N193K and L211M or L211E. Other advantageous phytase mutants showing an advantageous thermostability comprise the mutations D149N, S189D, K190N, K191N, K25E or K25A, G26S or G26N, N193H, N193S, N193R or N193K, R210Q and L211M. Particularly suitable phytase mutants according to the present invention showing an advantageous thermostability and particularly good phosphate release comprise the mutations D149N, S189D, K190N, K191N, K25E, G26S or G26N, N193S, N193R or N193K, R210Q and L211M. The bio-efficacious phytase mutant of the present invention is active for example up to 90° C. and active for example at a pH from 3 to 5.5.

The bio-efficacious phytase mutant of the present invention is used for example for the production of a food or feed product for example for humans and/or animals.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

The following figures show the invention in more detail, however, the invention is not limited to these figures.

FIG. 1: Improvement of 3-phytase mutants of SEQ ID NO: 2 regarding their catalytic performance in an application setup and their IC₅₀ values as a measure for thermostability; •wildtype SEQ ID NO: 2, •3-phytase mutants of SEQ ID: NO: 47, SEQ ID: NO: 4, SEQ ID: NO: 6, SEQ ID: NO: 7, SEQ ID: NO: 8, SEQ ID: NO: 10, SEQ ID: NO: 14, SEQ ID: NO: 19, SEQ ID: NO: 20, SEQ ID: NO: 21, SEQ ID: NO: 26, SEQ ID: NO: 28, SEQ ID: NO: 29, SEQ ID: NO: 30, SEQ ID: NO: 31, SEQ ID: NO: 34, SEQ ID: NO: 1, SEQ ID: NO: 37, SEQ ID: NO: 38, SEQ ID: NO: 39, SEQ ID: NO: 40, SEQ ID: NO: 41, SEQ ID: NO: 45, and SEQ ID: NO: 46.

FIG. 2: pH profiles of wildtype SEQ ID NO:2 and 3-phytase mutants of SEQ ID: NO: 19, SEQ ID: NO: 20, SEQ ID: NO: 21, SEQ ID: NO: 28, SEQ ID: NO: 30, SEQ ID: NO: 34, SEQ ID: NO: 1 at pH 2 to 5.5.

FIG. 3: Bio-efficiency Index of 3-phytase mutants SEQ ID: NO: 1, SEQ ID: NO: 3, SEQ ID: NO: 4, SEQ ID: NO: 5, SEQ ID: NO: 6, SEQ ID: NO: 7, SEQ ID: NO: 8, SEQ ID: NO: 9, SEQ ID: NO: 10, SEQ ID: NO: 11, SEQ ID: NO: 12, SEQ ID: NO: 13, SEQ ID: NO: 14, SEQ ID: NO: 15, SEQ ID: NO: 16, SEQ ID: NO: 17, SEQ ID: NO: 18, SEQ ID: NO: 19, SEQ ID: NO: 20, SEQ ID: NO: 21, SEQ ID: NO: 22, SEQ ID: NO: 23, SEQ ID: NO: 24, SEQ ID: NO: 25, SEQ ID: NO: 26, SEQ ID: NO: 27, SEQ ID: NO: 28, SEQ ID: NO: 29, SEQ ID: NO: 30, SEQ ID: NO: 31, SEQ ID: NO: 32, SEQ ID: NO: 33, SEQ ID: NO: 34, SEQ ID: NO: 35, SEQ ID: NO: 36, SEQ ID: NO: 37, SEQ ID: NO: 38, SEQ ID: NO: 39, SEQ ID: NO: 40, SEQ ID: NO: 41, SEQ ID: NO: 42, SEQ ID: NO: 43, SEQ ID: NO: 44, SEQ ID: NO: 45, SEQ ID: NO: 46, SEQ ID: NO: 47 in comparison to SEQ ID NO:2.

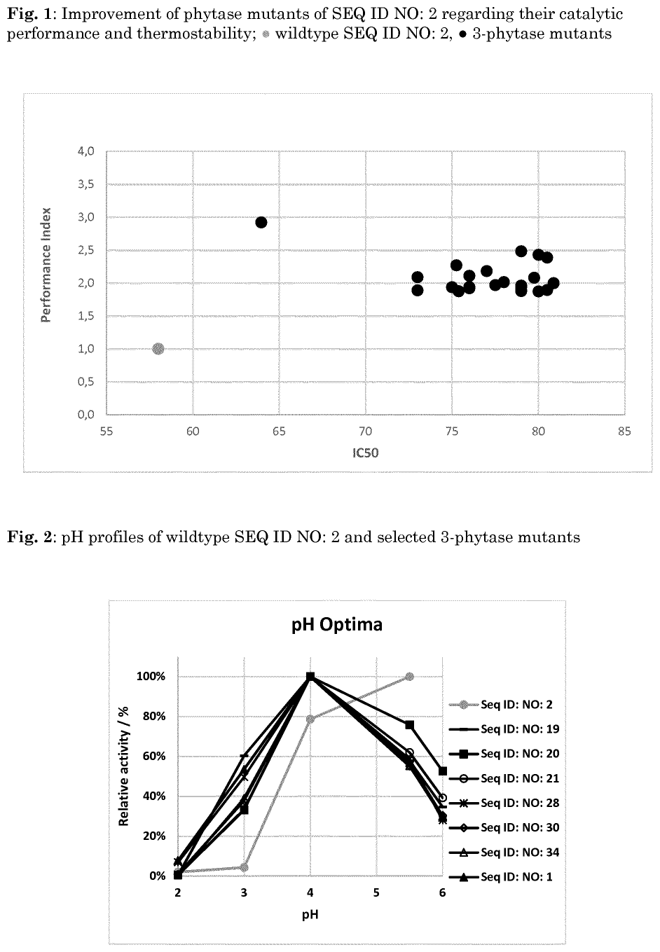

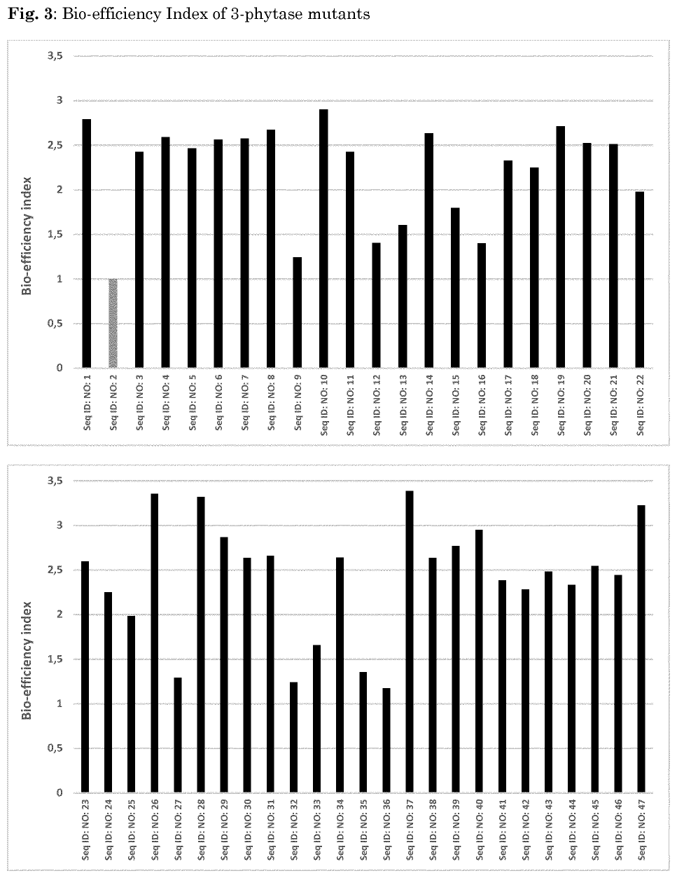

### DETAILED DESCRIPTION

The present invention is directed to a phytase mutant, such as a 3-phytase mutant having sequence identity to SEQ ID NO: 1 of 90% to 97%, 91% to 96%, 92% to 95%, at least 90%, at least 91%, at least 92%, at least 93%, at least 94%, at least 95%, at least 96.25% or at least 97% comprising or consisting of the sequence of SEQ ID NO.2 and one or more, e.g., at least two, at least three, at least four, at least five, at least six, at least seven, at least eight, at least nine or at least ten mutations.

Within the present invention, the term “phytase mutant” is to be understood as referring to any sequence which does not as such occur in nature but is the result of a genetic modification of at least one amino acid compared to a natural occurring phytase enzyme sequence.

The mutation of the phytase such as the 3-phytase of the present invention is selected from the group consisting of D149N, S189D, K190N, K191N, R210Q, L211E, L211M, K25E, K25A, G26N, G26S, N193H, N193K, N193S, N193R, D1E, P3T, G26K, K29A, T30S, T30A, T30K, D36N, Q45N, Q45R, V58E, S75C, Q76R, P107S, A112G, D120K, T129K, Q136R, Q144L, D149N, K187E, D189N, G192R, S195R, S195Y, S195T, S195N, N204I, N204T, N204S, S214A, N216D, A230T, M237L, N243K, N248K, R255H, G257S, F264Y, A275P, D276G, K295Y, K295M, Q332D, A352S, V383M, R393H, A395T and combinations thereof. The combinations comprise one or more mutations.

The resulting phytase mutant is bio-efficacious, i.e., the phytase mutant is consistently active under varying conditions which are in the present invention high temperatures for example up to 90° C. and acidic to basic pH for example in the range from pH 2.5 to 5.5.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

The temperature for example of a food or feed product and thus, of a phytase mutant for example contained in the food or feed product varies significantly with a storage temperature for example of −18° C., 4° C. or room temperature, the temperature in a subject such as a human or animal which is for example 37° C., and the temperature for the preparation of food or feed product for example in decontamination processes with temperatures, e.g., of up to 90° C. The bio-efficacious phytase mutant of the present invention is stable and active from 4° C., 10° C., 15° C., 20° C., 25° C., 28° C., 30° C., 35° C., 37° C., 40° C., 45° C., 50° C., 55° C., 60° C., 65° C., 70° C., or 75° C. to 90° C., or is stable and active up to 40° C., 45° C., 50° C., 55° C., 60° C., 65° C., 70° C., 75° C., 80° C., 85° C. or 90° C.

In addition, a phytase passing through the intestinal tract of a human or an animal is exposed to different pH ranges for example in humans the saliva has a pH of about 6.5 to 7.5, the upper stomach has a pH of about 4.0 to 6.5, the lower stomach has a pH of about 1.5 to 4.0, the duodenum has a pH of about 7.0 to 8.5 and the small and large intestine have a pH of about 4.0 to 7.0. A highly efficient phytase mutant such as a 3-phytase mutant resulting in a high cleavage rate of phosphate from a phytate has to be active in a broad pH range. The bio-efficacious phytase mutant of the present invention is stable and active in a range from pH 2.5 to 5.5, from pH 3.0 to 5.0, from pH 3.5 to 4.5, from 3.0 to 5.5 or from pH 5.0 to 5.5 and for example at any of the previously disclosed temperatures and temperature ranges, respectively.

The activity of a phytase and the phytase mutants of the present invention is determined by measuring the phosphate release which is indicated in mmol/L phytate at a specific pH. The Performance Index, which provides information about the activity of the phytase mutant at different pH in comparison to a reference such as SEQ ID NO: 2, is defined as [(Released phosphate [mM] at pH X)ₘᵤₜₐₙₜ/released phosphate [mM] at pH X)_{reference}]×[(released phosphate [mM] at pH Y)ₘᵤₜₐₙₜ/(released phosphate [mM] at pH Y)_{referenc}], wherein X and Y are different pH values, i.e., for example [(Released phosphate [mM] at pH 3)ₘᵤₜₐₙₜ/released phosphate [mM] at pH 3)_{SEQ ID NO 2}]×[(released phosphate [mM] at pH 5)ₘᵤₜₐₙₜ (released phosphate [mM] at pH 5)_{SEQ ID NO 2}].

The Bio-efficiency Index determines the correlation of performance and thermostability of a phytase mutant in comparison to a reference, e.g., SEQ ID NO.2, and is defined as IC₅₀₍ᵥₐᵣᵢₐₙₜ₎/IC_{50(reference)}×[(Released phosphate [mM] at pH X)ₘᵤₜₐₙₜ/released phosphate [mM] at pH X)_{reference}]×[(released phosphate [mM] at pH Y)ₘᵤₜₐₙₜ/(released phosphate [mM] at pH Y)_{reference}], wherein X and Y are different pH values, i.e., for example IC₅₀₍ᵥₐᵣᵢₐₙₜ₎/IC_{50(SEQ ID NO: 2)}×[(Released phosphate [mM] at pH 3)ₘᵤₜₐₙₜ/released phosphate [mM] at pH 3)_{SEQ ID NO: 2}]×[(released phosphate [mM] at pH 5)ₘᵤₜₐₙₜ/(released phosphate [mM] at pH 5)_{SEQ ID NO: 2}].

The term “feed” or “feed product” as used according to the present invention pertains to any product known to a person skilled in the art as suitable for feeding any kind of animal, for example mammals and birds, such as but not limited to ruminants, pigs, deer, poultry but also comprises fish or other aquatic livestock generally referred to as seafood. Included are feed products for non-ruminant animals, e.g. poultry, broilers, birds, chickens, turkeys, ducks, geese, and fowl; ruminant animals e.g. cows, cattle, horses, and sheep; pigs, swine, piglets, growing pigs, and sows; companion animals including but not limited to: cats, dogs, rodents, and rabbits; fish including but not limited to salmon, trout, tilapia, catfish and carp; and crustaceans including but not limited to shrimp and prawn.

The term “food” or “food product” as used according to the present invention pertains to any product known to a person skilled in the art as suitable for human consumption.

The feed or food product of the present invention for example contains starch. The starch-containing feed components typically include vegetable material such as cereal(s), e.g., one or more of corn (maize), wheat, barley, rye, rice, sorghum and millet, and/or tubers such as potatoes, cassava and sweet potato. The vegetable material may be milled, e.g., wet or dry milled grain, or distillers dry grain solids.

The feed or food product of the present invention for example contains protein-rich feed ingredients such as soybean (preferably soybean meal), rapeseed, palm kernel, cotton seed and sunflower.

Further, the feed or food product comprises for example at least one compound selected from the group consisting of vitamins, minerals, organic acids, probiotic components, oils, fats, pigments, growth factors and antimicrobial agents.

Optionally the feed or food product comprises at least one or more enzymes in addition to the 3-phytase mutant, for example food or feed enzymes which improve the digestibility of the feed and/or degrade intermediate products produced by the 3-phytase mutant such as inositol-tetraphosphate, e.g. another phytase such as 6-phytase, an amylase or a protease, aminopeptidase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidogluta-minase, peroxidase, polyphenoloxidase, proteolytic enzyme, ribonuclease, transglutaminase, or xylanase. The food or feed enzyme(s) are for example derived from microorganisms such as bacteria or fungi or from plants or animals.

The feed or food product is for example in form of a granulate, compactate, extrudate or liquid form.

The phytase mutant of the present invention is administered to a subject for example in a food or feed product, or directly without a food or feed product for example in form of a tablet, powder, liquid, an extrudate or granulate such as a coated granulate, e.g. with a coating comprising a salt, e.g. at least 60% w/w of the salt for example selected from the group consisting of NaCl, KCl, Na₂S0₄, K₂S0₄ and MgS0₄.

The phytase mutant of the present invention is used for example for the production of a food or feed product such as pelleted or extrudated mixtures of grains, corn, hay, molasses and straw for example in dry or liquid form.

As used herein “liquid form” pertains to a composition for example comprising a buffer, a stabilizer, and an anti-microbial agent and optionally at least one further enzyme, wherein the enzyme is an amylase, a cellulase, a lactase, a lipase, a protease, a catalase, a xylanase, a beta-glucanase, a mannanase, an amylase, an amidase, an epoxide hydrolase, an esterase, a phospholipase, a transaminase, an amine oxidase, a cellobiohydrolase, an ammonia lyase, or any combination thereof.

As used herein “dry form” pertains to a composition for example comprising one or more of the following components: a carrier, a buffer, a stabilizer, a binding agent, a plasticizer, an anti-microbial agent. Dry forms of a food or feed product comprise for example sucrose, sodium chloride, sorbitol, sodium citrate, potassium sorbate, sodium benzoate, sodium propionate, guar gum and/or wheat flour.

Sequences of phytase mutants of the present invention are for example the following, which were partially tested in the Examples:

SEQ ID NO: 1
DTPSSGWTLEKVVEVSRHGVRPPTENNVKTIQEGTNRQWPTWLTQYGELTG
HGYAAAVLKGHYEGQYLRQHGLLCQACPAPGDVFVWASPLQRTRETAMALM
DGVFSGCGVAIQGPEDEDNDPLFHAEKAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 2
DTPSSGWTLEKVVEVSRHGVRPPTKGNVKTIQEGTDRQWPTWLTQYGELTG
HGYAAAVLKGHYEGQYLRQHGLLSQACPAPGDVFVWASPLQRTRETAMALM
DGVFPGCGVAIQGPEDEDNDPLFHAETAGVTLDQQQVKADLQNAMQDKSAA
QIQAQWQPAIDRLKQAVCLPDKPCPAFSTPWEVKESKKGNISLHGPEVLAN
IAETIRLSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 3
DTPSSGWTLEKVVEVSRHGVRPPTENNVKTIQEGTNRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAMMKRLNDAVDRTALLPVHYTPHHHHH
H
SEQ ID NO: 4
DTPSSGWTLEKVVEVSRHGVRPPTESNVKTIQEGTNRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 5
DTPSSGWTLEKVVEVSRHGVRPPTENNVKTIQEGTNRQWPTWLTQYGELTG
HGYAAAVLKGHYEGQYLRQHGLLCQACPAPGDVFVWASPLQRTRETAMALM
DGVFSGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 6
DTPSSGWTLEKVVEVSRHGVRPPTESNVKTIQEGTDRQWPTWLTRYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 7
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLLNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 8
DTPSSGWTLEKVVEVSRHGVRPPTESNVKTIQEGTNRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDHTALLPVHYTPHHHHH
H
SEQ ID NO: 9
DTPSSGWTLEKVVEVSRHGVRPPTESNVKTIQEGTDRQWPTWLTQYGELTG
HGYAAAVLKGHYEGQYLRQHGLLSQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 10
DTPSSGWTLEKVVEVSRHGVRPPTESNVKTIQEGTDRQWPTWLTQYGELTG
HGYAAAVLKGHYEGQYLRQHGLLCRACPAPGDVFVWASPLQRTRETAMALM
DGVFPGCGVAIQGPEDEDNDPLFHAETAGVTLDRQQVKADLQNAMQNKSAA
QIQAQWQPAIDRLKQAVCLPDKPCPAFSTPWEVKEDNNGSISLHGPEVLAT
IAETIQMSYSNNNPLSEVAFGHAKTAAEVAALMPLLTANYDYTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 11
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNRSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 12
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNRSISLHGPEVLAI
IAETIQMSYSNNNPLSEVAFGHAKTAAEVAALMPLLTANYDFTNDLPYVAH
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 13
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIRMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 14
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTTAEVAALMPLLTANYDFTKDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTTLLPVHYTPHHHHH
H
SEQ ID NO: 15
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIRMSYSNNNPLSEVAFGHAKTAAEVAALMPLLTANYDFTNDLPYVAR
RSASVLMNQIALALNTEHQAGAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 16
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGHISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 17
DTPSSGWTLEKVVEVSRHGVRPPTESNVKTIQEGTDRQWPTWLTRYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQPDAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 18
DTPSSGWTLEKVVEVSRHGVRPPTESNVKTIQEGTDRQWPTWLTNYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 19
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 20
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTKDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 21
DTPSSGWTLEKVVEVSRHGVRPPTESNVKSIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTKDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 22
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIRMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLASLDDSH
PPLRSEFSMPDCQKTDIGTLCPYDAVMKRLNDAVDRTALLPVHYTPHHHHH
H
SEQ ID NO: 23
DTPSSGWTLEKVVEVSRHGVRPPTEKNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSISLHGPEVLAI
IAETIRESYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 24
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEKNDPLFHAETAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 25
DTPSSGWTLEKVVEVSRHGVRPPTESNVKAIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 26
DTPSSGWTLEKVVEVSRHGVRPPTESNVKEIQEGTDRQWPTWLTQYGELTG
HGYAAAVLKGHYEGQYLRQHGLLCQACPAPGDVFVWASPLQRTRETAMALM
DGVFPGCGVAIQGPEDEKNDPLFHAETAGVTLDQQQVKADLQNAMQNKSAA
QIQAQWQPAIDRLKQAVCLPDKPCPAFSTPWEVKEDNNGKIYLHGPEVLAS
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 27
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 28
DTPSSGWTLEKVVEVSRHGVRPPTESNVKTIQEGTNRQWPTWLTQYGELTG
HGYAAAVLKGHYEGQYLRQHGLLCQACPAPGDVFVWASPLQRTRETAMALM
DGVFPGCGVAIQGPEDEKNDPLFHAETAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 29
DTPSSGWTLEKVVEVSRHGVRPPTESNVKAIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 30
DTPSSGWTLEKVVEVSRHGVRPPTESNVKTIQEGTNRQWPTWLTQYGELTG
HGYAAAVLKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 31
DTPSSGWTLEKVVEVSRHGVRPPTESNVKTIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 32
DTPSSGWTLEKVVEVSRHGVRPPTENNVKTIQEGTNRQWPTWLTQYGELTG
HGYAAAVLKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 33
DTPSSGWTLEKVVEVSRHGVRPPTASNVKKIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGHISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 34
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGRIYLHGPEVLAN
IAETIQMSYSNNNPLSEVAFGHAKTAAEVAALMPLLTAKYDFTNDLPYVAR
RGASVLMNQIALALNTEHQADAPPDAKWLLYVAHDTNIAYLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 35
DTPSSGWTLEKVVEVSRHGVRPPTENNVKTIQEGTNRQWPTWLTQYGELTG
HGYAAAVLKGHYEGQYLRQHGLLCQACPAPGDVFVWASPLQRTRETAMALM
DGVFSGCGVAIQGPEDEDNDPLFHAEKAGVTLDQQQVKADLQNAMQNKSAA
QIQAQWQPAIDRLKQAVCLPDKPCPAFSTPWEVKEDNNGSISLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 36
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAEKAGVTLDQQQVKADLQNAMQNKSAA
QIQAQWQPAIDRLKQAVCLPDKPCPAFSTPWEVKEDNNGKIYLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 37
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIQMSYSNNNPLSEVAFGHAKTAAEVAALLPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 38
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVGIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIQMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 39
DTPSSGWTLEKVVEVSRHGVRPPTASNVAKIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSITLHGPEVLAI
IAETIQMSYSNNNPLSEVAFGHAKTAAEVAALMPLLTANYDFTKDLPYVAR
RGASVLMNQIALALNTEHQADAPPDAKWLLYVAHDTNIAMLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVRYTPHHHHH
H
SEQ ID NO: 40
DTPSSGWTLEKVVEVSRHGVRPPTASNVAKIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVEEDNNGSISLHGPEVLAI
IAETIQMSYSNNNPLSEVAFGHAKTAAEVAALMPLLTANYDFTKDLPYVAR
RGASVLMNQIALALNTEHQADAPPDAKWLLYVAHDTNIAMLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 41
ETTSSGWTLEKVVEVSRHGVRPPTASNVAKIQEGTDRQWPTWLTQYGELTG
HGYAAAVLKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSITLHGPEVLAN
IAETIQMSYSNDNPLSEVAFGHAKTAAEVAALMPLLTAKYDFTNDLPYVAR
RGASVLMNQIALALNTEHQADAPPDAKWLLYVAHDTNIAMLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVRYTPHHHHH
H
SEQ ID NO: 42
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIQMSYANNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 43
DTPSSGWTLEKVVEVSRHGVRPPTEGNVKAIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIQMSYSNNNPLSEVAFGHAKTAAEVAALMPLLTANYDFTKDLPYVAR
RGASVLMNQIALALNTEHQADAPPDAKWLLYVAHDTNIAKLRTMLGFTWQM
GDYPRGNIPPAGSLIFERWRNQQSGDRVVRIYFQAQSLDQIRGLAALDDSH
PPLRSEFSMPDCQKTDIGTLCPYDAVMKRLNDAVDRTALLPVHYTPHHHHH
H
SEQ ID NO: 44
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIRMSYSNNNPLSEVAFGHAKTAAEVAALMPLLTANYDFTNDLPYVAR
RGASVLMNQIALALNTEHQADAPPDAKWLLYVAHDTNIAKLRTMLGFTWQM
GDYPRGNIPPAGSLIFERWRNQQSGQRVVRIYFQAQSLDQIRGLAALDDSH
PPLRSEFSMPDCQKTDIGTLCPYDAVMKRLNDAVDRTALLPVHYTPHHHHH
H
SEQ ID NO: 45
DTPSAAAPSSGWTLEKVVEVSRHGVRPPTESNVKTIQEGTDRQWPTWLTQY
GELTGHGYAAAELKGHYEGQYLRQHGLLCQACPAPGDVFVWASPLQRTRET
AMALMDGVFPGCGVAIQGPEDEDNDPLFHAETAGVTLDQQQVKADLQNAMQ
NKSAAQIQAQWQPAIDRLKQAVCLPDKPCPAFSTPWEVKEDNNGSIRLHGP
EVLAIIAETIQMSYSNNNPLSEVAFGHAKTAAEVAALMPLLTANYDFTNDL
PYVARRGASVLMNQIALALNTEHQADAPPDAKWLLYVAHDTNIAKLRTMLG
FTWQMGDYPRGNIPPAGSLIFERWRNQQSGQRVVRIYFQAQSLDQIRGLAA
LDDSHPPLRSEFSMPDCQKTDIGTLCPYDAVMKRLNDAVDRTALLPVHYTP
HHHHHH
SEQ ID NO: 46
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVLAI
IAETIQMSYSNNNPLSEVAFGHAKTAAEVAALMPLLTANYDFTNDLPYVAR
RGASVLMNQIALALNTEHQAAAAQADAPPDAKWLLYVAHDTNIAKLRTMLG
FTWQMGDYPRGNIPPAGSLIFERWRNQQSGQRVVRIYFQAQSLDQIRGLAA
LDDSHPPLRSEFSMPDCQKTDIGTLCPYDAVMKRLNDAVDRTALLPVHYTP
HHHHHH
SEQ ID NO: 47
DTPSSGWTLEKVVEVSRHGVRPPTESNVKTIQEGTDRQWPTWLTQYGELTG
HGYAAAELKGHYEGQYLRQHGLLCQACPAPGDVFVWASPLQRTRETAMALM
DGVFPGCGVAIQGPEDEDNDPLFHAETAGVTLDQQQVKADLQNAMQNKSAA
QIQAQWQPAIDRLKQAVCLPDKPCPAFSTPWEVKEDNNGSIRLHGPEVAAA
EVLAIIAETIQMSYSNNNPLSEVAFGHAKTAAEVAALMPLLTANYDFTNDL
PYVARRGASVLMNQIALALNTEHQADAPPDAKWLLYVAHDTNIAKLRTMLG
FTWQMGDYPRGNIPPAGSLIFERWRNQQSGQRVVRIYFQAQSLDQIRGLAA
LDDSHPPLRSEFSMPDCQKTDIGTLCPYDAVMKRLNDAVDRTALLPVHYTP
HHHHHH

### EXAMPLES

In the following the present invention is described by the examples. The examples are considered for illustrative purpose only and do not limit the scope of the present invention and claims in any respect.

### Example 1: Thermostability of 3-Phytase Mutants

Characterization of 3-phytase mutants regarding their thermostability was done by measuring the volumetric activity at pH 5.5 in acetate buffer before and after heat treatment. Heat treatment was done for 15 minutes at different temperatures (58 to 85° C.) and the enzymatic activity was determined by measuring the phosphate release at different time points using the photometric assay according to Example 4. Using the activities before and after heat treatment IC₅₀ values were determined, wherein IC₅₀ is defined as the temperature at which the enzyme shows 50% residual activity after 25 min. of incubation. All 3-phytase mutants of SEQ ID NO: 2 show significantly higher thermostability than the wildtype SEQ ID NO: 2 as presented in the following:

| SEQ ID NO | IC₅₀ [° C.] |
|---|---|
| SEQ ID. NO: 2 | 58 |
| SEQ ID. NO: 1 | 81 |
| SEQ ID. NO: 3 | 80 |
| SEQ ID. NO: 4 | 80 |
| SEQ ID. NO: 5 | 79 |
| SEQ ID. NO: 6 | 79 |
| SEQ ID. NO: 7 | 79 |
| SEQ ID. NO: 8 | 79 |
| SEQ ID. NO: 9 | 78 |
| SEQ ID. NO: 10 | 77 |
| SEQ ID. NO: 11 | 77 |
| SEQ ID. NO: 12 | 74 |
| SEQ ID. NO: 13 | 73 |
| SEQ ID. NO: 14 | 73 |
| SEQ ID. NO: 15 | 71 |
| SEQ ID. NO: 16 | 79 |
| SEQ ID. NO: 17 | 79 |
| SEQ ID. NO: 18 | 79 |
| SEQ ID. NO: 19 | 78 |
| SEQ ID. NO: 20 | 76 |
| SEQ ID. NO: 21 | 75 |
| SEQ ID. NO: 22 | 74 |
| SEQ ID. NO: 23 | 73 |
| SEQ ID. NO: 24 | 79 |
| SEQ ID. NO: 25 | 79 |
| SEQ ID. NO: 26 | 80 |
| SEQ ID. NO: 27 | 78 |
| SEQ ID. NO: 28 | 81 |
| SEQ ID. NO: 29 | 80 |
| SEQ ID. NO: 30 | 81 |
| SEQ ID. NO: 31 | 79 |
| SEQ ID. NO: 32 | 80 |
| SEQ ID. NO: 33 | 79 |
| SEQ ID. NO: 34 | 79 |
| SEQ ID. NO: 35 | 79 |
| SEQ ID. NO: 36 | 79 |
| SEQ ID. NO: 37 | 79 |
| SEQ ID. NO: 38 | 78 |
| SEQ ID. NO: 39 | 76 |
| SEQ ID. NO: 40 | 75 |
| SEQ ID. NO: 41 | 73 |
| SEQ ID. NO: 42 | 76 |
| SEQ ID. NO: 43 | 75 |
| SEQ ID. NO: 44 | 73 |
| SEQ ID. NO: 45 | 76 |
| SEQ ID. NO: 46 | 75 |
| SEQ ID. NO: 47 | 64 |

### Example 2: Phosphate Release in Application Testing of Thermostable 3-Phytase Mutants Comprising the Mutations D149N, S189D, K190N, K191N, R210Q and L211M

The phosphate release of each 3-phytase mutant of SEQ ID NO: 2 comprising the mutations D149N, S189D, K190N, K191N, R210Q and L211M was determined in a close-to-application testing and compared to the wildtype SEQ ID NO:2. Therefore, 3-phytase mutants were expressed in Pichia pastoris and the volumetric activity was determined according to DIN EN ISO 30024:2009 (https://www.iso.org/standard/45787.html) with minor changes (format downscaled by a factor of 5). The enzymes were applied with 500 units/kg feed in the application testing. Here, 15 g of a 70/30 mixture of corn and soy was incubated at 41° C. using 25% dry matter for 60 minutes at pH 3 and 5. Afterwards, the released phosphate was measured using the ammonium molybdate assay according to Example 4. The Performance Index was calculated as follows: [(Released phosphate [mM] at pH 3)ᵥₐᵣᵢₐₙₜ/released phosphate [mM] at pH 3)_{SEQ ID NO: 2}]×[(released phosphate [mM] at pH 5)ᵥₐᵣᵢₐₙₜ/(released phosphate [mM] at pH 5)_{SEQ ID NO: 2}]. All 3-phytase mutants show an improved performance index compared to the wildtype SEQ ID NO: 2 as presented in the following Table:

| SEQ ID NO | Performance Index |
|---|---|
| SEQ ID. NO: 2 | 1.00 |
| SEQ ID. NO: 47 | 2.92 |
| SEQ ID. NO: 4 | 1.88 |
| SEQ ID. NO: 6 | 1.88 |
| SEQ ID. NO: 7 | 1.89 |
| SEQ ID. NO: 8 | 1.96 |
| SEQ ID. NO: 10 | 2.18 |
| SEQ ID. NO: 14 | 2.09 |
| SEQ ID. NO: 19 | 2.02 |
| SEQ ID. NO: 20 | 1.93 |
| SEQ ID. NO: 21 | 1.94 |
| SEQ ID. NO: 26 | 2.43 |
| SEQ ID. NO: 28 | 2.39 |
| SEQ ID. NO: 29 | 2.08 |
| SEQ ID. NO: 30 | 1.90 |
| SEQ ID. NO: 31 | 1.95 |
| SEQ ID. NO: 34 | 1.94 |
| SEQ ID. NO: 1 | 2.00 |
| SEQ ID. NO: 37 | 2.49 |
| SEQ ID. NO: 38 | 1.97 |
| SEQ ID. NO: 39 | 2.11 |
| SEQ ID. NO: 40 | 2.27 |
| SEQ ID. NO: 41 | 1.89 |
| SEQ ID. NO: 45 | 1.94 |
| SEQ ID. NO: 46 | 1.88 |

### Example 3: Determination of the pH Profile of Thermostable 3-Phytase Mutants of SEQ ID NO: 2

The improved performance of the 3-phytase mutants is majorly founded by a shifted pH profile towards a lower pH. Therefore, the pH profile of selected 3-phytase mutants of SEQ ID NO: 2 was determined by measuring the activity at different pH values using the photometric assay according to Example 4. Fig. shows a shift in pH optimum for all variants compared to the wildtype SEQ ID NO: 2.

### Example 4: Determination of the Enzymatic Activity of 3-Phytase Mutants

5 μL enzyme solution (in 100 mmol/L sodium acetate buffer, pH 5.5, 0.05% (w/v) Triton X-100) were incubated with 95 μL 2.88 mmol/L sodium phytate (Sigma 68388, lot BCBM4006V) in 100 mmol/L sodium acetate buffer, pH 5.5 at for 25 min. The reaction was stopped by adding 100 μL 10% (w/v) trichloroacetic acid. Subsequently, 100 μL of the stopped enzymatic reaction was mixed with 100 μL molybdate reagent (aqueous solution of 1.2% (w/v) ammonium molybdate; 4.4% (v/v) sulfuric acid, and 27 mg/mL (w/v) ferrous sulfate) and the solution was incubated for 15 min at room temperature. Absorbance at 700 nm was determined and the amount of released inorganic phosphate was calculated using inorganic phosphate standard solutions (0-1.5 mmol/L). One unit of phytase activity was defined as the amount of enzyme that releases 1 μmol phosphate per min at 37° C.

1. Bio-efficacious phytase mutant having sequence identity of at least 96.25% around SEQ ID NO. 2, wherein the mutant comprises a sequence of SEQ ID NO. 2 and a mutation selected from the group consisting of D149N, S189D, K190N, K191N, R210Q, L211E, L211M, K25E, K25A, G26N, G26S, N193H, N193K, N193S, N193R, D1E, P3T, G26K, K29A, T30S, T30A, T30K, D36N, Q45N, Q45R, V58E, S75C, Q76R, P107S, A112G, D120K, T129K, Q136R, Q144L, D149N, K187E, D189N, G192R, S195R, S195Y, S195T, S195N, N204I, N204T, N204S, S214A, N216D, A230T, M237L, N243K, N248K, R255H, G257S, F264Y, A275P, D276G, K295Y, K295M, Q332D, A352S, V383M, R393H, A395T and combinations thereof.

2. Bio-efficacious phytase mutant according to claim 1 comprising mutations D149N, S189D, K190N and K191N.

3. Bio-efficacious phytase mutant according to claim 1 comprising mutations R210Q and L211M.

4. Bio-efficacious phytase mutant according to claim 1 comprising mutations K25E or K25A, and G26N or G26S, and N193S or N193R.

5. Bio-efficacious phytase mutant according to claim 1 comprising mutations at positions 25, 26, 193, 210 and/or 211.

6. Bio-efficacious phytase mutant according to claim 1 comprising mutations at positions D149N, S189D, K190N, K191N, K25E or K25A, G26S or G26N, N193S, N193R or N193K, R210Q and L211M.

7. Bio-efficacious phytase mutant according to claim 1, which is active up to 90° C.

8. Bio-efficacious phytase mutant according to claim 1, which is active at a pH from 3 to 5.5.

9. Use of the bio-efficacious phytase mutant according to claim 1 for the production of a food or feed product.


SEQUENCE LISTING (47 sequences)

SEQ ID NO: 1 - protein, 409 aa, Artificial Sequence
  Variant of wt Sequence (Seq2)
DTPSSGWTLEKVVEVSRHGVRPPTENNVKTIQEGTNRQWPTWLTQYGELTGHGYAAAVLKGHYEGQYLRQHGLLCQACPAPGDVFVWASPLQRTRETAMALMDGVFSGCGVAIQGPEDEDNDPLFHAEKAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 2 - protein, 409 aa, Artificial Sequence
  wt sequence of Dickeya zeae phytase with His tag
DTPSSGWTLEKVVEVSRHGVRPPTKGNVKTIQEGTDRQWPTWLTQYGELTGHGYAAAVLKGHYEGQYLRQHGLLSQACPAPGDVFVWASPLQRTRETAMALMDGVFPGCGVAIQGPEDEDNDPLFHAETAGVTLDQQQVKADLQNAMQDKSAAQIQAQWQPAIDRLKQAVCLPDKPCPAFSTPWEVKESKKGNISLHGPEVLANIAETIRLSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 3 - protein, 409 aa, Artificial Sequence
  Variant of wt sequence (Seq2)
DTPSSGWTLEKVVEVSRHGVRPPTENNVKTIQEGTNRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAMMKRLNDAVDRTALLPVHYTPHHHHHH

SEQ ID NO: 4 - protein, 409 aa, Artificial Sequence
  Variant of wt sequence (Seq2)
DTPSSGWTLEKVVEVSRHGVRPPTESNVKTIQEGTNRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 5 - protein, 409 aa, Artificial Sequence
  Variant of wt sequence (Seq2)
DTPSSGWTLEKVVEVSRHGVRPPTENNVKTIQEGTNRQWPTWLTQYGELTGHGYAAAVLKGHYEGQYLRQHGLLCQACPAPGDVFVWASPLQRTRETAMALMDGVFSGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 6 - protein, 409 aa, Artificial Sequence
  Variant of wt sequence (Seq2)
DTPSSGWTLEKVVEVSRHGVRPPTESNVKTIQEGTDRQWPTWLTRYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 7 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLLNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 8 - protein, 409 aa, Artificial Sequence
  Variant of wt sequence (Seq2)
DTPSSGWTLEKVVEVSRHGVRPPTESNVKTIQEGTNRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDHTALLPVHYTPHHHHHH

SEQ ID NO: 9 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLSQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 10 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCRACPAPGDVFVWASPLQRTRETAMALMDGVFPGCGVAIQGPEDEDNDPLFHAETAGVTLDRQQVKADLQNAMQNKSAAQIQAQWQPAIDRLKQAVCLPDKPCPAFSTPWEVKEDNNGSISLHGPEVLATIAETIQMSYSNNNPLSEVAFGHAKTAAEVAALMPLLTANYDYTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 11 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNRSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 12 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNRSISLHGPEVLAIIAETIQMSYSNNNPLSEVAFGHAKTAAEVAALMPLLTANYDFTNDLPYVAHRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 13 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIRMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 14 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTTAEVAALMPLLTANYDFTKDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTTLLPVHYTPHHHHHH

SEQ ID NO: 15 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIRMSYSNNNPLSEVAFGHAKTAAEVAALMPLLTANYDFTNDLPYVARRSASVLMNQIALALNTEHQAGAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 16 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGHISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 17 - protein, 409 aa, Artificial Sequence
  Variant of wt sequence (Seq2)
DTPSSGWTLEKVVEVSRHGVRPPTESNVKTIQEGTDRQWPTWLTRYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQPDAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 18 - protein, 409 aa, Artificial Sequence
  Variant of wt sequence (Seq2)
DTPSSGWTLEKVVEVSRHGVRPPTESNVKTIQEGTDRQWPTWLTNYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 19 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 20 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTKDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 21 - protein, 409 aa, Artificial Sequence
  Variant of wt sequence (Seq2)
DTPSSGWTLEKVVEVSRHGVRPPTESNVKSIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTKDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 22 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIRMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLASLDDSHPPLRSEFSMPDCQKTDIGTLCPYDAVMKRLNDAVDRTALLPVHYTPHHHHHH

SEQ ID NO: 23 - protein, 409 aa, Artificial Sequence
  Variant of wt sequence (Seq2)
DTPSSGWTLEKVVEVSRHGVRPPTEKNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSISLHGPEVLAIIAETIRESYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 24 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEKNDPLFHAETAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 25 - protein, 409 aa, Artificial Sequence
  Variant of wt sequence (Seq2)
DTPSSGWTLEKVVEVSRHGVRPPTESNVKAIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 26 - protein, 409 aa, Artificial Sequence
  Variant of wt sequence (Seq2)
DTPSSGWTLEKVVEVSRHGVRPPTESNVKEIQEGTDRQWPTWLTQYGELTGHGYAAAVLKGHYEGQYLRQHGLLCQACPAPGDVFVWASPLQRTRETAMALMDGVFPGCGVAIQGPEDEKNDPLFHAETAGVTLDQQQVKADLQNAMQNKSAAQIQAQWQPAIDRLKQAVCLPDKPCPAFSTPWEVKEDNNGKIYLHGPEVLASIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 27 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 28 - protein, 409 aa, Artificial Sequence
  Variant of wt sequence (Seq2)
DTPSSGWTLEKVVEVSRHGVRPPTESNVKTIQEGTNRQWPTWLTQYGELTGHGYAAAVLKGHYEGQYLRQHGLLCQACPAPGDVFVWASPLQRTRETAMALMDGVFPGCGVAIQGPEDEKNDPLFHAETAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 29 - protein, 409 aa, Artificial Sequence
  Variant of wt sequence (Seq2)
DTPSSGWTLEKVVEVSRHGVRPPTESNVKAIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 30 - protein, 409 aa, Artificial Sequence
  Variant of wt sequence (Seq2)
DTPSSGWTLEKVVEVSRHGVRPPTESNVKTIQEGTNRQWPTWLTQYGELTGHGYAAAVLKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 31 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 32 - protein, 409 aa, Artificial Sequence
  Variant of wt sequence (Seq2)
DTPSSGWTLEKVVEVSRHGVRPPTENNVKTIQEGTNRQWPTWLTQYGELTGHGYAAAVLKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 33 - protein, 409 aa, Artificial Sequence
  Variant of wt sequence (Seq2)
DTPSSGWTLEKVVEVSRHGVRPPTASNVKKIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGHISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 34 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGRIYLHGPEVLANIAETIQMSYSNNNPLSEVAFGHAKTAAEVAALMPLLTAKYDFTNDLPYVARRGASVLMNQIALALNTEHQADAPPDAKWLLYVAHDTNIAYLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 35 - protein, 409 aa, Artificial Sequence
  Variant of wt sequence (Seq2)
DTPSSGWTLEKVVEVSRHGVRPPTENNVKTIQEGTNRQWPTWLTQYGELTGHGYAAAVLKGHYEGQYLRQHGLLCQACPAPGDVFVWASPLQRTRETAMALMDGVFSGCGVAIQGPEDEDNDPLFHAEKAGVTLDQQQVKADLQNAMQNKSAAQIQAQWQPAIDRLKQAVCLPDKPCPAFSTPWEVKEDNNGSISLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 36 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAEKAGVTLDQQQVKADLQNAMQNKSAAQIQAQWQPAIDRLKQAVCLPDKPCPAFSTPWEVKEDNNGKIYLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 37 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYSNNNPLSEVAFGHAKTAAEVAALLPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 38 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVGIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 39 - protein, 409 aa, Artificial Sequence
  Variant of wt sequence (Seq2)
DTPSSGWTLEKVVEVSRHGVRPPTASNVAKIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSITLHGPEVLAIIAETIQMSYSNNNPLSEVAFGHAKTAAEVAALMPLLTANYDFTKDLPYVARRGASVLMNQIALALNTEHQADAPPDAKWLLYVAHDTNIAMLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVRYTPHHHHHH

SEQ ID NO: 40 - protein, 409 aa, Artificial Sequence
  Variant of wt sequence (Seq2)
DTPSSGWTLEKVVEVSRHGVRPPTASNVAKIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVEEDNNGSISLHGPEVLAIIAETIQMSYSNNNPLSEVAFGHAKTAAEVAALMPLLTANYDFTKDLPYVARRGASVLMNQIALALNTEHQADAPPDAKWLLYVAHDTNIAMLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 41 - protein, 409 aa, Artificial Sequence
  Variant of wt sequence (Seq2)
ETTSSGWTLEKVVEVSRHGVRPPTASNVAKIQEGTDRQWPTWLTQYGELTGHGYAAAVLKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSITLHGPEVLANIAETIQMSYSNDNPLSEVAFGHAKTAAEVAALMPLLTAKYDFTNDLPYVARRGASVLMNQIALALNTEHQADAPPDAKWLLYVAHDTNIAMLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVRYTPHHHHHH

SEQ ID NO: 42 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYANNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 43 - protein, 409 aa, Artificial Sequence
  Variant of wt sequence (Seq2)
DTPSSGWTLEKVVEVSRHGVRPPTEGNVKAIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYSNNNPLSEVAFGHAKTAAEVAALMPLLTANYDFTKDLPYVARRGASVLMNQIALALNTEHQADAPPDAKWLLYVAHDTNIAKLRTMLGFTWQMGDYPRGNIPPAGSLIFERWRNQQSGDRVVRIYFQAQSLDQIRGLAALDDSHPPLRSEFSMPDCQKTDIGTLCPYDAVMKRLNDAVDRTALLPVHYTPHHHHHH

SEQ ID NO: 44 - protein, 409 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIRMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 45 - protein, 414 aa, Artificial Sequence
  Variant of wt sequence (Seq2)
DTPSAAAPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 46 - protein, 414 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVLAIIAETIQMSYSNNNPLSEVAFGHAKTAAEVAALMPLLTANYDFTNDLPYVARRGASVLMNQIALALNTEHQAAAAQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH

SEQ ID NO: 47 - protein, 414 aa, Artificial Sequence
  Variant of wt sequence (Seq2)
DTPSSGWTLEKVVEVSRHGVRPPTESNVKTIQEGTDRQWPTWLTQYGELTGHGYAAAELKGHYEGQYLRQHGLLCQACPAPGDVFVWASPLQRTRETAMALMDGVFPGCGVAIQGPEDEDNDPLFHAETAGVTLDQQQVKADLQNAMQNKSAAQIQAQWQPAIDRLKQAVCLPDKPCPAFSTPWEVKEDNNGSIRLHGPEVAAAEVLAIIAETIQMSYSNNNPLSEVAFGHAKTAAEVAALMPLLTANYDFTNDLPYVARRGASVLMNQIALALNTEHQADAPPDAKWLLYVAHDTNIAKLRTMLGFTWQMGDYPRGNIPPAGSLIFERWRNQQSGQRVVRIYFQAQSLDQIRGLAALDDSHPPLRSEFSMPDCQKTDIGTLCPYDAVMKRLNDAVDRTALLPVHYTPHHHHHH